(19) United States
(12) Patent Application Publication
(10) Pub. No.: US 2011/0300155 A1
(43) Pub. Date: Dec. 8, 2011

(54) MODULATING XRN2

(75) Inventors: Saibal Chatterjee, West Bengal (IN); Helge Grosshans, Basel (CH)

(73) Assignee: NOVARTIS FORSCHUNGSSTIFTUNG, ZWEIGNIEDERLASSUNG, F RIEDRICH MIESCHER INSTITUTE FOR BIOMWDICAL RESEAR, Basel (CH)

(21) Appl. No.: 13/132,657

(22) PCT Filed: Dec. 3, 2009
(86) PCT No.: PCT/EP2009/066287

§ 371 Date: Aug. 26, 2011

(30) Foreign Application Priority Data
Dec. 4, 2008 (EP) 08170721.8

(51) Int. Cl. A61K 39/395; C12Q 1/68; A61P 31/12; A61P 3/00; A61P 9/00; C12N 9/99; A61K 31/713

(52) U.S. Cl. 424/158.1; 435/184; 435/6.1; 435/6.11; 514/44 A

(57) ABSTRACT

The present invention relates to a method for modulating miRNA, said method being characterized in that a modulator of XRN2 is used. Also provided are uses of said method for therapeutical purposes, reagents therefore, as well as screening methods.

### FIELD OF THE INVENTION

The present invention relates to a method of modulating miRNA through XRN2.

### BACKGROUND OF THE INVENTION

Animal miRNAs repress their targets through an antisense mechanism, where they base-pair imperfectly with their target mRNAs, promoting translational repression and target degradation (Filipowicz 2008). While some miRNAs are expressed at a constant steady-state level during animal development, others exhibit very dynamic expression patterns (Lim 2003, Houbaviy 2003, Neilson 2007), imparted by both transcriptional and post-transcriptional regulation (Martinez 2008, Thomson 2006, Obernosterer 2006), which occurs at various steps of miRNA biogenesis. As RNA concentrations are generally a function of biogenesis and turnover, the inventors considered that it would possible that active miRNA degradation can also modulate miRNA accumulation, providing an additional layer of regulation of miRNA activity. Recent studies have implicated miRNA mis-expression in various human diseases such as cancers and indicate that miRNAs can function as tumour suppressors and oncogenes (Chang 2007, Esquela-Kerscher 2006). miRNAs have also been shown to repress the expression of important cancer-related genes and might prove useful in the diagnosis and treatment of cancer (Chang 2007, Esquela-Kerscher 2006). Thus understanding of miRNA turnover would not only provide new insights into miRNA metabolism circuit but would also open up new avenues towards unravelling of these pathological states.

### SUMMARY OF THE INVENTION

Using genetic and biochemical approaches in C. elegans, the present inventors have now surprisingly identified and characterized a 5′-to-3′ exonuclease; xrn-2 or XRN2, as a component of miRNA turnover machinery. Their results indicate that degradation occurs on mature miRNAs, after they have been incorporated into miRNA-induced silencing complex (miRISC), and that degradation by XRN2 is therefore an extremely important part of the miRNA metabolism circuit.

The present invention therefore encompasses a method for modulating miRNA, said method being characterized in that a modulator of XRN2 is used. In some embodiments of the invention, the method of the invention is performed in a subject and wherein an effective amount of said modulator of XRN2 is administered to said subject. For instance, the method is performed to treat a disease in a subject and a therapeutically effective amount of said modulator of XRN2 is administered to said subject. Examples of diseases, for which the methods of the present invention are relevant, are cancers, metabolic diseases, developmental disorders, cardiac diseases or viral infections.

In some embodiments of the invention, the modulator of XRN2 is a small molecule, for instance a RNase inhibitor, a siRNA or an antibody. The modulator of XRN2 according to the present invention can be either an agonist or an antagonist (inhibitor), which might decrease or silence the expression of XRN2, depending of the scope of the method and of the miRNA to be modulated.

In some embodiments of the invention, the subject is a mammal, for instance a human.

The present invention also encompasses a siRNA decreasing or silencing XRN2 and/or an antibody specifically binding to XRN2, for use as a medicament.

The present invention also encompasses a method for the identification of a substance that modulates the expression of XRN2 and/or its biological activity, which method comprises the steps of (i) contacting a XRN2 polypeptide or a fragment thereof having the biological activity of XRN2, a polynucleotide encoding such a polypeptide or polypeptide fragment, an expression vector comprising such a polynucleotide or a cell comprising such an expression vector, and a test substance under conditions that in the absence of the test substance would permit XRN2 expression and/or biological activity; and (ii) determining the amount of expression and/or biological activity of XRN2, to determine whether the test substance modulates biological activity and/or expression of XRN2, wherein a test substance which modulates biological activity and/or expression of the XRN2 is a potential therapeutical agent to treat cancer. In some embodiments, the biological activity of XRN2 is the degradation of mature miRNA.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

FIG. 1. Depletion of xrn-2 increases miRNA levels and activity in vivo

A), B) Northern blotting of RNA from let-7(n2853) worms exposed to the indicated RNAi. xrn-2(RNAi) leads to the accumulation of mature let-7 comparable to the levels in wild-type (N2) worms. mir-85 and mir-77 also accumulate relative to empty vector RNAi (control) but mature lin-4 or pre-mir-60 do not. tRNA^{Gly} serves as loading control.

C) Examination of total RNA by RT-qPCR analysis demonstrates that xrn-2(RNAi) does not cause an increase of pri-let-7 or pri-mir-77 levels relative to control.

D) RT-qPCR demonstrates that the levels of lin-41, daf-12 mRNAs, two let-7 targets are elevated in let-7(n2853) relative to N2 and let-7(n2853); xrn-2(RNAi) animals

All experiments in this and subsequent figures used synchronized L4 stage animals. Results in C and D are averages of three independent experiments +/−standard error of mean.

FIG. 2. Xrn-2 is required for miRNA degradation in vitro.

Radiolabelled mature let-7 miRNA was incubated with lysate from N2 worms, the reaction products were analyzed by urea PAGE unless indicated otherwise.

A) Fate of 3′-pCp end labelled and blocked synthetic let-7 exposed to lysate at two different temperatures: 25° C., worm physiological temperature; 37° C., conventional biochemical assay temperature. An identical, single product (mononucleotide) is obtained at both the temperatures, although 37° C. incubation yielded more product. All subsequent in vitro assays were performed at 37° C.

B) Incubation of 5′-end labelled synthetic let-7 with lysate also yields mononucleotides.

C) Thin layer chromatographic analysis of the assay performed with internally α-³²P-UTP labelled let-7 and lysate. 5′-UMP co-migrated with the assay product (shown by arrowhead on top right), whereas 5′-UDP migrated much slower.

D) 5′-end labelled yeast tRNA^{Phe} was incubated with lysate. An array of bands (indicated by a vertical bar on the right) migrating slower than the input RNA and a final product of a few nucleotides length (indicated by an arrowhead on the left) were obtained.

E) Incubation of 3′-end labelled and blocked synthetic let-7 (lane 1) with control lysate (lane 2), xrn-2(RNAi) lysate (lane 3), xrn-2(RNAi) lysate supplemented with recombinant GST-XRN-2 (lane 4), xrn-2(RNAi) lysate supplemented with GST only (lane 5) shows that xrn-2 is required for miRNA turnover.

F) xrn-1(RNAi) lysate and control RNAi lysates support degradation of 3′-end labelled and blocked synthetic let-7 equally efficiently.

G) 5′-end phosphorylation is required for efficient degradation of a 3′-end labelled and blocked synthetic let-7 RNA in larval lysate.

FIG. 3. Coordination of in vitro miRNA processing and turnover.

In vitro assays were performed with α-³²P-UTP/cold UTP labelled in vitro transcribed pre-let-7 and N2 worm lysate, unless indicated otherwise.

A) Schematic representation of the stepwise processing of pre-let-7/pre-miRNA stem-loop into single stranded mature miRNA.

B) Fate of radiolabelled pre-let-7 incubated with lysate. The products are shown with arrowheads on the left and some relevant sizes have been indicated on the right.

C) Pre-let-7 is stabilized in dcr-1 (RNAi) relative to control RNAi lysates.

D) Northern blot confirms accumulation of endogenous pre-let-7 in dcr-1 (RNAi) animals in vivo, confirming efficient dicer depletion. tRNA^{Gly} levels serve as loading control.

E) Pre-let-7 assayed with control and xrn-2(RNAi) lysate. Left side arrowheads point to different reaction products, which include one band corresponding to mature let-7 in size (right side arrowhead.

F) A 20-fold scaled-up pre-let-7 turnover assay was performed with cold substrate and micrococcal nuclease (MN) treated lysates as indicated. The recovered products were subjected to northern probing for let-7, revealing accumulation of mature let-7 (arrowhead).

G) 5′-end labelled let-7 guide RNA annealed to the passenger strand was incubated with lysate subsequently analyzed on a native gel, demonstrating its stability. 5′-end labelled single stranded (ss) let-7 migrates faster than double stranded (ds) 5′-labelled guide-passenger duplex.

H) Pre let-7 turnover assay was performed with control and xrn-2(RNAi) lysates. Native gel analysis shows that the main band accumulating in xrn-2(RNAi) lysates co-migrates with single stranded mature let-7. Asterisk points to a conformer of pre-let-7.

FIG. 4. Target mediated stabilization of mature miRNA

A) Schematic representation of Renilla luciferase (RL) reporter mRNAs, containing synthetic 3′UTRs docking a) three functional let-7 binding sites, b) three mutated sites and c) lacking a 3′ UTR.

B) Pre-let-7 assay with radiolabelled substrate and N2 lysate in absence or presence of three different mRNAs as indicated; reveals accumulation of (pointed by arrowhead) a band corresponding to mature let-7 in size.

C) The above assay was performed in a 20 fold higher scale with cold substrates and MN-treated lysates, eliminating endogenous RNAs. Recovered RNA was subjected to northern probing, demonstrating accumulation of mature let-7 in the presence of its target RNA.

D) Pre-let-7 turnover assay (top panel) was performed as above except control and xrn-2 kd lysates were used from a gfp::ago expressing strain. Middle panel shows the immunoprecipitates from the corresponding top panel reactions using α-GFP antibody, and the lower panel shows the recovered material from the post-immunoprecipitation supernatant (sup).

FIG. 5. Active release of miRNA from miRISC argonautes

A) Equal amounts of immunoprecipitated miRISC argonaute (GFP/AGO) proteins were subjected, while bead-bound, to either no treatment (lane 1), or incubated at 37° C. for 15 min with assay buffer (AB, lane 2), AB+high salt (lane 3), and the same amount of lysate from which the miRISC argonautes (used in the reaction) have been immunoprecipitated (lane 4). After recovery each reaction was split into two halves. One half was subjected to RNA extraction followed by northern probing with α-let-7 probe (upper panel), and the corresponding other halves (lower panel) were subjected to western blotting using α-GFP antibody.

B) Immunoprecipitation was performed as above from lysate either directly or after incubation at 25° C. for 15 min. One half of the immunoprecipitate was subjected to northern probing (upper panel) after RNA extraction, and the corresponding other halves (lower panel) were subjected to western blotting as above. Lane 3 corresponds to a no-antibody control.

### DETAILED DESCRIPTION OF THE INVENTION

Using genetic and biochemical approaches in C. elegans, the present inventors have now surprisingly identified and characterized a 5′-to-3′ exonuclease; xrn-2 or XRN2, as a component of miRNA turnover machinery. Their results indicate that degradation occurs on mature miRNAs, after they have been incorporated into miRNA-induced silencing complex (miRISC), and that degradation by XRN2 is therefore an extremely important part of the miRNA metabolism circuit.

With their work, the inventors have shown here that XRN-2 is required for miRNA turnover in vivo and in vitro, and that it can terminate the activity of functional miRNAs in vivo. Thus, the inventors demonstrated that miRNA degradation contributes to miRNA homeostasis, helping to prevent detrimental overexpression of miRNAs associated with disease. Reflecting their complementary roles in the maintenance of proper gene expression, inventors also found that mature miRNA biogenesis and turnover are coordinated in vitro.

The present inventors have also observed that miRNA targets can stabilize their cognate miRNAs in vitro, potentially permitting coordination of miRNA levels with abundance of their targets. Under conditions of reduced target abundance, such a mechanism makes Argonaute available for loading of other miRNAs, facilitating its reuse. Additionally, when miRNA silencing is relieved or prevented by antagonists such as HuR or Dnd1 (Bhattacharyya 2006, Kedde 2008), increased degradation of unoccupied miRNA can provide a mechanism that enhances desilencing by preventing the miRISC from re-binding its released target, thus restricting cycles of alternate silencing and desilencing.

The present invention therefore encompasses a method for modulating miRNA, said method being characterized in that a modulator of XRN2 is used. In some embodiments of the invention, the method of the invention is performed in a subject and wherein an effective amount of said modulator of XRN2 is administered to said subject. In some embodiments, the subject is a non-human animal, for instance for scientific research purposes. In some other embodiments, the method is performed to treat a disease in a subject and a therapeutically effective amount of said modulator of XRN2 is administered to said subject. Examples of diseases, for which the methods of the present invention are relevant, are cancers, metabolic diseases, developmental disorders, cardiac diseases or viral infections. In some embodiments, the diseases, for which the methods of the present invention are relevant, are selected for a group of diseases comprising glioblastoma, breast cancer, cholangiocarcinoma, chronic lymphocytic leukemia (CLL), colorectal neoplasia, diffuse large B cell lymphoma (DLBCL), head and neck cancer, hepatocellular carcinoma, lung cancer, lymphomas, ovarian cancer, pancreatic cancer, papillary thyroid carcinoma, pituitary adenomas, prostate cancer, stomach cancer, testicular germ cell tumours, diabetes, dis-regulated lipid metabolism, increased plasma cholesterol levels, HIV infection, EBV infection, HCMV infection, HCV infection, cardiac hypertrophy, Alzheimer's disease, psoriasis, PFV-1 infection, Tourette's syndrome (TS), Parkinson's disease and schizophrenia.

In some embodiments of the invention, the modulator of XRN2 is a small molecule, for instance a RNase inhibitor, a siRNA or an antibody. The modulator of XRN2 according to the present invention can be either an agonist, which might increase or initiate the expression of XRN2 and/or its biological activity, or an antagonist (inhibitor), which might decrease or silence the expression of XRN2 and/or its biological activity, depending of the scope of the method and of the miRNA to be modulated. For example, an agonist of XRN2 would increase its degradative action on mature miRNA, for instance by blocking the binding site for a negative regulator of XRN2, whereas an antagonist could block the enzymatic activity of XRN2, for instance by occupying its active site.

In some embodiments of the invention, the subject is a mammal, for instance a human. In some other embodiment, the subject is a non-human animal or organism. Examples of such non-human animal or organism are rats, mice, yeasts, flies, worms, plants, bacteria, insects, isolated cells, and the like.

The present invention also encompasses a siRNA decreasing or silencing XRN2 and/or an antibody specifically binding to XRN2, for use as a medicament.

The present invention also encompasses a method for the identification of a substance that modulates the expression of XRN2 and/or its biological activity, which method comprises the steps of (i) contacting a XRN2 polypeptide or a fragment thereof having the biological activity of XRN2, a polynucleotide encoding such a polypeptide or polypeptide fragment, an expression vector comprising such a polynucleotide or a cell comprising such an expression vector, and a test substance under conditions that in the absence of the test substance would permit XRN2 expression and/or biological activity; and (ii) determining the amount of expression and/or biological activity of XRN2, to determine whether the test substance modulates biological activity and/or expression of XRN2, wherein a test substance which modulates biological activity and/or expression of the XRN2 is a potential therapeutical agent to treat cancer. In some embodiments, the biological activity of XRN2 is the degradation of mature miRNA.

In addition, the present invention also encompasses the modulators of the expression of expression and/or of its biological activity of XRN2 identified using a method of screening of the invention.

Another embodiment of the invention encompasses the use of a XRN2 as a biomarker for cancers or developmental disorders. In this embodiment, the expression level or protein concentration of XRN2 is measured in a sample from a subject and compared to the expression level or protein concentration in a normal subject, wherein said normal subject can be a pool of subjects, and wherein an up- or down-regulation of XRN2 is indicative of a possible cancer or developmental dysfunction, or risk therefor.

Moreover, in some embodiments of the invention the modulators of XRN2 are used to control and regulate, either positively or negatively, the action of siRNA introduced into cells and targeted to any target.

These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment”, “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, derivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W.H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty-1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=1, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

In one embodiment where one is assaying for the ability to bind or compete with full-length XRN2 polypeptide for binding to XRN2 antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Assays described herein and otherwise known in the art may routinely be applied to measure the ability of XRN2 polypeptides and fragments, variants derivatives and analogs thereof to elicit XRN2-related biological activity (either in vitro or in vivo).

The term “epitopes,” as used herein, refers to portions of a polypeptide having antigenic or immunogenic activity in an animal, in some embodiments, a mammal, for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno-specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CH1, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgGl, IgG2, IgG3, IgG4, IgAl and IgA2) or subclass of immunoglobulin molecule.

In addition, in the context of the present invention, the term “antibody” shall also encompass alternative molecules having the same function, e.g. aptamers and/or CDRs grafted onto alternative peptidic or non-peptidic frames.

In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CHI, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues.

Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof.

Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide are also included in the present invention.

Antibodies may also be described or specified in terms of their binding affinity to a polypeptide

Antibodies may act as agonists or antagonists of the recognized polypeptides. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(I):14-20 (1996).

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity.

Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide).

Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat cancer. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

The invention also provides methods for treating cancer in a subject by inhibiting XRN2 by administration to the subject of an effective amount of an inhibitory compound or pharmaceutical composition comprising such inhibitory compound. In some embodiments, said inhibitory compound is an antibody or an siRNA. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-13 8 (1984)).

Other controlled release systems are discussed in the review by Langer (Science 249:1527-1533 (1990)).

The present invention also provides pharmaceutical compositions for use in the treatment of cancer by inhibiting XRN2. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems.

For antibodies, the dosage administered to a patient is typically 0.1 mg/kg to 100 mg/kg of the patient's body weight. In some embodiments, the dosage administered to a patient is between 0.1 mg/kg and 20 mg/kg of the patient's body weight, for example 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown’ of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid.

The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Frequently, automatically generated alignments require manual alignment, exploiting the trained users knowledge of the protein family being studied, e.g., biological knowledge of key conserved sites. One such alignment editor programs is Align (http://www.gwdg.de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYL1P.

The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the inhibitor is a siRNA molecule and comprises between approximately 5 bp and 50 bp, in some embodiments, between 10 bp and 35 bp, or between 15 bp and 30 bp, for instance between 18 bp and 25 bp. In some embodiments, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among:
a) a 3′ cap;
b) a 5′ cap,
c) a modified internucleoside linkage; or
d) a modified sugar or base moiety.

Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates.

End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference.

The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918.

The reaction may be carried out in solution or, in some embodiments, on solid phase or by using polymer supported reagents, followed by combining the synthesized RNA strands under conditions, wherein a siRNA molecule is formed, which is capable of mediating RNAi.

It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesise the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like).

Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly.

In an embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product).

Inhibitors according to any embodiment of the present invention may be used in a monotherapy (e.g. use of siRNAs alone). However it will be appreciated that the inhibitors may be used as an adjunct, or in combination with other therapies.

The modulators of XRN2 may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site.

The modulators of XRN2 may be used in a number of ways.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted at the site of a tumour, and the molecule may be released over weeks or months. Such devices may be particularly advantageous when long term treatment with a modulator of XRN2 is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration.

Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an modulator of XRN2 may be used for the treatment of cancer in the subject, depending upon which specific inhibitor is used. When the inhibitor is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the inhibitor (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

The term “cancer” refers to a group of diseases in which cells are aggressive (grow and divide without respect to normal limits), invasive (invade and destroy adjacent tissues), and sometimes metastatic (spread to other locations in the body). These three malignant properties of cancers differentiate them from benign tumors, which are self-limited in their growth and don't invade or metastasize (although some benign tumor types are capable of becoming malignant). A particular type of cancer is a cancer forming solid tumours. Such cancer forming solid tumours can be breast cancer, prostate carcinoma or oral squamous carcinoma. Other cancer forming solid tumours for which the methods and inhibitors of the invention would be well suited can be selected from the group consisting of adrenal cortical carcinomas, angiomatoid fibrous histiocytomas (AFH), squamous cell bladder carcinomas, urothelial carcinomas, bone tumours, e.g. adamantinomas, aneurysmal bone cysts, chondroblastomas, chondromas, chondromyxoid fibromas, chondrosarcomas, fibrous dysplasias of the bone, giant cell tumours, osteochondromas or osteosarcomas, breast tumours, e.g. secretory ductal carcinomas, chordomas, clear cell hidradenomas of the skin (CCH), colorectal adenocarcinomas, carcinomas of the gallbladder and extrahepatic bile ducts, combined hepatocellular and cholangiocarcinomas, fibrogenesis imperfecta ossium, pleomorphic salivary gland adenomas head and neck squamous cell carcinomas, chromophobe renal cell carcinomas, clear cell renal cell carcinomas, nephroblastomas (Wilms tumor), papillary renal cell carcinomas, primary renal ASPSCR1-TFE3 t(X;17)(p11;q25) tumors, renal cell carcinomas, laryngeal squamous cell carcinomas, liver adenomas, hepatoblastomas, hepatocellular carcinomas, non-small cell lung carcinomas, small cell lung cancers, malignant melanoma of soft parts, medulloblastomas, meningiomas, neuroblastomas, astrocytic tumours, ependymomas, peripheral nerve sheath tumours, neuroendocrine tumours, e.g. phaeochromocytomas, neurofibromas, oral squamous cell carcinomas, ovarian tumours, e.g. epithelial ovarian tumours, germ cell tumours or sex cord-stromal tumours, pericytomas, pituitary adenomas, posterior uveal melanomas, rhabdoid tumours, skin melanomas, cutaneous benign fibrous histiocytomas, intravenous leiomyomatosis, aggressive angiomyxomas, liposarcomas, myxoid liposarcomas, low grade fibromyxoid sarcomas, soft tissue leiomyosarcomas, biphasic synovial sarcomas, soft tissue chondromas, alveolar soft part sarcomas, clear cell sarcomas, desmoplastic small round cell tumours, elastofibromas, Ewing's tumours, extraskeletal myxoid chondrosarcomas, inflammatory myofibroblastic tumours, lipoblastomas, lipoma, benign lipomatous tumours, liposarcomas, malignant lipomatous tumours, malignant myoepitheliomas, rhabdomyosarcomas, synovial sarcomas, squamous cell cancers, subungual exostosis, germ cell tumours in the testis, spermatocytic seminomas, anaplastic (undifferentiated) carcinomas, oncocytic tumours, papillary carcinomas, carcinomas of the cervix, endometrial carcinomas, leiomyoma as well as vulva and/or vagina tumours. In an embodiment of the invention, the cancer is a cancer of the pancreas, large intestine, small intestine, lungs or ovary. In another embodiment, the cancer is a cancer of the brain, for instance an astrocytoma, a glioblastoma or an oligodendroglioma.

In one embodiment, the cancer is a XRN2-dependent cancer. XRN2-dependent cancers are cancers where XRN2 has become an essential gene. XRN2-dependent cancers can be easily identified by depleting the cells of XRN2 expression, and identifying the cancers that are not able to grow in the absence of XRN2.

Developmental disorders are disorders that occur at some stage in a child's development, often retarding the development. These may include psychological or physical disorders. Examples of developmental disorders are developmental disabilities, mental retardation, learning disabilities, neurodevelopmental disorders, specific developmental disorders or pervasive developmental disorders.

Examples of metabolic diseases include, but are not limited to, metabolic syndrome (also known as “Syndrome X”), impaired glucose tolerance, impaired fasting glucose, hypercholesterolemia, hyperlipidemia, hypertriglyceridemia, low HDL levels, hypertension, phenylketonuria, post-prandial lipidemia, a glycogen-storage disease, Gaucher's Disease, Tay-Sachs Disease, Niemann-Pick Disease, ketosis and acidosis.

As reviewed in a recent article by Zhang & Farwell (J. Cell. Mol. Med. Vol 12, No. 1, 2008 pp. 3-21), more and more evidence indicates that miRNAs play an important role in many human diseases, ranging widely from cancers, HIV to metabolic diseases. This evidence includes, but not limited to, (1) a unique set of miRNAs exists in a specific disease; (2) a unique expression of miRNAs in a certain human disease and (3) aberrant expression of miRNAs in human disease.

For instance, it has been demonstrated that almost all cancers have alternative miRNA expression profile compared to their adjunct normal tissues. These cancer types include several important cancers, for example lung cancer, leukaemia, brain cancer and breast cancer, which together cause the majority of cancer-related death in the past decades. Interestingly, recent studies also demonstrated tumour invasion and metastasis is also initiated by miRNAs. Moreover, several studies demonstrated that miRNAs have an important function in metabolism and in metabolic diseases. Furthermore, it is also well-known that miRNAs are important to control viral replication when the virus infects a cell and to further control virus infection. In addition to the diseases reviewed above, miRNAs also regulate several other diseases. For example, aberrant expression of miRNAs are associated with several neuronal diseases, including Tourette's syndrome, Alzheimer's disease, schizophrenia and schizoaffective disorder. Recently, several investigations demonstrated that miRNAs play an important role in cardiac development and contractility, and several heart diseases are associated with the aberrant expression of certain miRNAs.

The present invention also provides a method of screening compounds to identify those which might be useful for treating cancer in a subject by inhibiting XRN2, as well as the so-identified compounds.

XRN2, also termed xrn-2, XRN-2, EC 3.1.13.-, 5′-3′ exoribonuclease 2, DHM1-like protein, HP protein, or Dhm1-like protein (mouse homolog) has the Entrez Gene ID: 22803. It is an exoribonuclease enzyme which possesses 5′->3′ exoribonuclease activity. This gene shares similarity with the mouse Dhm1 and the yeast dhp1 gene. The yeast gene is involved in homologous recombination and RNA metabolism, such as RNA synthesis and RNA trafficking. Complementation studies show that Dhm1 has a similar function in mouse as dhp1.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

Worm strains, let-7(n2853) suppression and RNAi. The wild type strain was C. elegans var. Bristol strain N2. The other two strains were let-7(n2853) (Reinhart), gfp::alg-1;gfp::alg-2 (Hutvagner). Suppressors of let-7(n2853) were identified by RNAi growing worms at 25° C. as described (Grosshans 2005, Ding 2008)

RNA isolation, Northern blotting and RT-qPCR. Total RNA was isolated from staged worms using Trizol™ (Invitrogen) method as described before (Lee & Ambros 2001). Northern blotting of endogenous RNA was done as described in reference (Pall 2008). 5′-labelled (using T4 polynucleotide kinase [PNK] and γ-³²P-ATP) DNA oligos were used as probes. However, the hybridization for let-7 miRNA was carried out at an elevated temperature of 40° C. in order to minimize the binding of the probe to let-7 sisters. For northern analysis of in vitro turnover assay products, the lysates were pre-treated with micrococcal nuclease (MN, NEB, 0.5 μl/10-20 μg of lyaste) for 10 min at 37° C. followed by addition of EGTA to a final concentration of 7.5 mM. MN pre-treatment was done to digest all endogenous RNAs from the lysate ruling out the possibility of highlighting any endogenous RNA. Excess EGTA was used to terminate the MN treatment.—EGTA lysate served as a positive control for MN activity, which was sufficient to remove all the RNA, including exogenous RNA; resulting in no signal. After assay-reaction incubation; the samples were phenol-chloroform extracted and alcohol precipitated. The recovered samples were subjected to northern probing as per conditions stated above. RT-qPCR was carried out using kits from Promega (RT) and Thermo Scientific (qPCR) following the manufacturer's instructions and employing oligos furnished below.

Cloning and expression of recombinant Xrn-2. xrn-2 cDNA was amplified from total RNA through RT-PCR, cloned in a TOPO TA vector (Invitrogen), and confirmed through sequencing. The ORF was subcloned in pGEX 4T-1 (GE Healthcare) and expressed in E. coli as glutathione S-transferase (GST) fusion protein. The recombinant protein was detergent extracted from inclusion bodies and then resolved on SDS PAGE. After KCl staining (Hager 1980), the band of pure recombinant protein was excised, and the protein was eluted with buffer PEB (0.05 M Tris-HCl [pH 8.0], 0.2 M NaCl, 0.1 mM EDTA, 5 mM DTT, 0.2% SDS) overnight at 37° C. The eluate was stored as such in aliquots at −80° C. Before use the recombinant protein was refolded by diluting 20 fold in TETN 250 buffer containing 0.1% Triton X 100 (Chatterjee 2006), and incubated for 2 h at 4° C. Finally the protein was microcon-100 (Millipore) concentrated and estimated through Bradford assay (Bio-Rad).

Preparation of RNA substrates. Pre-let-7 or mature let-7 RNA were prepared essentially following the methods described by Kolb et al. (Methods Enzymol, 2005). In brief, a chimeric RNA containing in its 5′ portion a hammerhead ribozyme followed by the pre let-7/mature let-7 sequence was transcribed from DNA cassettes using a T7 MAXIscript kit from Ambion, in presence of α-³²P-UTP or cold UTP. The DNA cassettes were prepared by annealing of appropriate forward and reverse primers followed by Klenow fill-in reactions. Double stranded DNAs of appropriate length were gel purified and PCR amplified using appropriate flanking primers. Gel purified PCR products were used as the templates for in vitro transcription reactions. Moreover, before use, the PCR products were cloned and sequence confirmed. Self-processing of the ribozyme-containing transcripts occur during the course of transcription reaction. The resulting pre-let-7/mature let-7 containing 5′ hydroxyl groups were size purified by 7 M urea/8-10% PAGE. After recovery RNAs were 5′ phosphorylated by T4 PNK and ATP. Before use the pre-let-7 RNA was subjected to refolding as described before (Kolb 2005). 5′ labelling of synthetic mature miRNAs and tRNA (yeast tRNA^{Phe}, Sigma) were done using PNK and γ ³²P ATP. 3′ labelling/blocking of synthetic mature miRNAs were done using T4 RNA ligase and [5′-³²P] pCp, according to manufacturers instructions (Ambion). All RNAs were gel purified. The 5′-7-methyl-G-capped RL reporter mRNAs were prepared through in vitro run-off transcription of appropriately restricted plasmids (Pillai 2005) using standard reagents from a T7 MEGAscript kit and cap analog m⁷ G(5′)ppp(5′)G from Ambion, as per the manufacturer's instruction. After phenol-chloroform extraction and alcohol precipitation the RNAs were polyadenylated using E. coli PolyA polymerase (Stratagene) and ATP.

Preparation of worm lysate. Staged worms grown on plates were harvested with M9 and washed thrice with the same buffer. The worm pellet was then resuspended in extraction buffer (10 mM HEPES [pH 7.4], 2 mM DTT, 0.1% Triton-X 100, 50 mM KCl, 0.5 mM PMSF, 10% Glycerol) and ground in liquid N₂. After thawing the sample was then spun at 14,000 g for 15 min, and the clear supernatant was collected, and designated as cleared worm lysate.

in vitro turnover assay. Labelled RNAs (pre-let-7 and mature let-7, approximately 1 and 2 fmol respectively) were incubated with cleared worm lysate (10-20 μg) in 1× assay buffer (AB; 10 mM HEPES [pH 7.4], 2 mM DTT, 5 mM MgCl₂, 100 mM KCl, 2 mM ATP) in a volume of 10 μl at 25° C./37° C. for 15 min. The reactions were stopped by addition of 1 volume Formamide gel loading buffer (95% Formamide, 0.2% SDS, 1 mM EDTA, 0.04% Xylene Cyanol, 0.04% Bromophenol Blue) followed by heating at 65° C. for 5 min. Equal volumes of the samples were then subjected to 7 M urea/8-12% PAGE followed by gel drying and autoradiography or phosphor-imaging. The target mRNA mediated miRNA stabilization assays were done with 1 fmol of radiolabelled substrate and 20 fmol of the concerned target mRNA in volumes of 20 μl. For addback assays, the KD lysate was pre-incubated with the recombinant protein (˜7 ng/reaction) on ice for 30 min in order to achieve reconstitution, and then used for the assay. For native gel analysis of pre-let-7 assay products, reactions were done as above except that after incubation the samples were subjected to Proteinase K (PK) treatment as described before (Matranga 2005) at room temperature for 30 min, and then resolved in a 15% native polyacrylamide gel at 4° C., using a native gel loading buffer (Matranga 2005).

Guide-passenger duplex with a 5′-³²P labelled guide strand was prepared using methods described before (Matranga 2005). 10,000 cpm of the native gel purified substrate was used per reaction. After incubation the reactions were subjected to PK treatment and alcohol precipitation as described before (Matranga 2005). The recovered samples were resuspended in a buffer as described before (Matranga 2005) and subjected to 15% native PAGE analysis at 4° C.

Coupled pre-let-7 processing and Ago immunoprecipitation. Pre-let-7 assay was performed as described above in absence or presence of target mRNA using a lysate obtained from a strain in which the C. elegans miRISC components; ALG-1 & ALG-2 (Ago) are tagged with GFP. After incubation for 15 min at 37° C., the reaction volumes were increased to 200 μl with 1×AB and subjected to immunoprecipitation at 4° C. for 2 hrs using α-GFP antibody (α-GFP mouse IgG; monoclonal antibody, Roche [Cat. # 11 814 460 001]) and protein A sepharose CL-4B (GE healthcare). The recovered sepharose beads were suspended in formamide gel loading buffer, heated at 65 C for 5 min, spun briefly and the sups were subjected to urea PAGE analysis. The post-immunoprecipitate sups were also recovered through phenol-chloroform extraction and alcohol precipitation, and subjected to urea PAGE analysis.

Thin Layer Chromatography. Mature miRNA turnover Reactions using α-³²P-UTP labelled miRNA were stopped by the addition of SDS to 1% and EDTA to 10 mM. Aliquots of 1 μl were spotted onto pre-washed PEI cellulose plates (Macherey-Nagel) and developed sequentially with 0.5 M LiCl and 1 M formic acid (Dziembowski 2007). Cold Uridine 5′ mono-, and di-phosphates were also separated on the same plate and visualized through fluorescence quenching.

miRNA dislodging/release assay. Immunoprecipitation of GFP-tagged ALG-1/ALG-2 complexes was essentially performed following the methods described by Lee and Schedl (Genes & Development, 2001) using the aforementioned α-GFP antibody. The bead-bound immunoprecipitates (derivative of 200 μg lysate protein was used per reaction) were then incubated with 1×AB, 1×AB plus KCl (to a final concentration of ˜0.6 M), or the same amount of lysate from which it has been immunoprecipitated, at 37° C. for 15 min. After further recovery the beads were split into two halves. From one half RNA was extracted for northern analysis and the other half was boiled in SDS sample buffer and subjected to SDS-PAGE and western blotting using the same α-GFP antibody used for IP, in order to confirm equal binding of Ago to the beads and integrity of the proteins.

In the parallel approach, immunoprecipitation was performed from lysate before and after incubation at the worm physiological temperature 25° C. for 15 min, and the immunoprecipitate was subjected to both northern and western probing as mentioned above.

Oligos (5′-3′).
Northern:
(SEQ ID NO: 1)
let-7(WT): AAC TAT ACA ACC TAC TAC CTC A;
(SEQ ID NO: 2)
let-7(n2853): AAC TAT ACA ACC TAC TAT CTC A;
(SEQ ID NO: 3)
mir-77: TGG ACA GCT ATG GCC TGA TGA A;
(SEQ ID NO: 4)
mir-85: GCA CGA CTT TTC AAA TAC TTT GTA;
(SEQ ID NO: 5)
lin-4: TCA CAC TTG AGG TCT CAG GGA;
(SEQ ID NO: 6)
pre mir-60: CT TGA ACT AGA AAA TGT GCA TAA TA TCA
CGT ACT TTG TCA TG;
(SEQ ID NO: 7)
5.8s rRNA: CAA CCC TGA ACC AGA CGT ACC AAC TGG AGG
CCC AGT TGG T;
(SEQ ID NO: 8)
tRNA ^{Gly}: GCTTGGAAGGCATCCATGCTGACCATT
qPCR:
Primary let-7:
(SEQ ID NO: 9)
Forward: TCCTAGAACACATCTCCCTTTGA;
(SEQ ID NO: 10)
Reverse: CGC AGC TTC GAA GAG TTC TG.
Primary mir-77:
(SEQ ID NO: 11)
Forward: CATTGTTCGTTTCGCTTTCA;
(SEQ ID NO: 12)
Reverse: CCA ATA ACT GAT TCA ACA TTC CAA.
daf-12 mRNA:
(SEQ ID NO: 13)
Forward: GAT CCT CCG ATG AAC GAA AA;
(SEQ ID NO: 14)
Reverse: CTC TTC GGC TTC ACC AGA AC.
lin-41 mRNA:
(SEQ ID NO: 15)
Forward: GGA TTG TTC GAC ACC AAC G;
(SEQ ID NO: 16)
Reverse: ACC ATG ATG TCA AAC TGC TGT C.
xrn-2 mRNA:
(SEQ ID NO: 17)
Forward: GAT CCC GAG TAC CCA CAA GA;
(SEQ ID NO: 18)
Reverse: CCA CCA CCA CCT CTC ACA TA.
Cloning:
xrn-2 cDNA
(SEQ ID NO: 19)
Forward Primer: GAAA GAATTC ATG GGA GTT CCC
GCA TTC TTC AG
(SEQ ID NO: 20)
Reverse primer: GAAA GCGGCCGC GAT TAT CTC CAT
GAT GAA TTT CCG TG
Preparation of templates for in vitro
transcription:
Mature let-7 cassette: Forward primer (T7
Promoter, HH Ribozyme, First 12 let-7 nt.)
(SEQ ID NO: 21)
G TAA TAC GAC TCA CTA TAG GGAGA CTA CTA CCT CAC
TGA TGA GTC CGT GAG GAC GAA ACG GTA CCC GGT ACC
GTC TGA GGT AGT AGG;
Reverse primer (Mature let-7 complementary
sequence, 12 nt. Complementary region to
HH Ribozyme)
(SEQ ID NO: 22)
AAC TAT ACA ACC TAC TAC CTC A GAC GGT ACC GGG.
Pre-/et-7 cassette: Forward primer (T7 Promoter,
HH Ribozyme, First 12 let-7 nt.)
(SEQ ID NO: 23)
G TAA TAC GAC TCA CTA TAG GGAGA CTA CTA CCT CAC
TGA TGA GTC CGT GAG GAC GAA ACG GTA ACCC GGT ACC
GTC TGA GGT GT AGG;
Reverse primer (Pre-/et-7 complementary sequence,
12 nt. Complementary region to HH Ribozyme)
(SEQ ID NO: 24)
GGT AAG GTA GAA AAT TGC ATA GTT CAC CGG TGG TAA
TAT TCC AAA CTA TAC AAC CTA CTA CCT CA GAC GGT
ACC GGG.
Primers for PCR amplification of mature
let-7 cassette: Forward T7 promoter primer
(SEQ ID NO: 25)
GAATTC TAA TAC GAC TCA CTA TAG G;
let-7 guide reverse primer
(SEQ ID NO: 26)
AAC TAT ACA ACC TAC TAC CTC A.
Primers for PCR amplification of pre-/et-7
cassette: Forward T7 promoter primer
(SEQ ID NO: 27)
GAATTC TAA TAC GAC TCA CTA TAG G;
let-7 passenger reverse primer
(SEQ ID NO: 28)
GGT AAG GTA GAA AAT TGC ATA G

Depletion of the xrn-2 exonuclease increases let-7 miRNA levels and activity in vivo. The let-7 miRNA regulates stem cell fates in animals and functions as a human tumor suppressor gene (Bussing 2008). In C. elegans, the temperature-sensitive let-7(n2853) causes vulval bursting phenotype at the larval-to-adult transition when let-7(n2853) animals are grown at the restrictive temperature, 25° C. This allele is characterized by a single point mutation towards the 5′ end of the mature miRNA, impairing its binding to target mRNAs (Reinhart 2000, Vella 2004). However, the expression levels of this mature miRNA are also moderately decreased compared to its wild-type counterpart (Reinhart 2000, Bagga 2005). As target site mutations compensatory to the let-7(n2853) point mutation restore target gene repression in a let-7(n2853) background only partially, this reduction appears functionally relevant (Vella 2004). The present inventors therefore hypothesized that increased expression of the mutant let-7 and/or its sequence-related ‘sister’ miRNAs mir-48, mir-84, and mir-241 (Abbott 2005) might be sufficient to downregulate some of its targets, thus partially suppressing the vulval bursting of let-7(n2853) animals

An RNAi-based screen of genes encoded on chromosome I had previously identified suppressors of the let-7(n2853) mutation, including known and novel let-7 target genes (Ding 2008). However, some of the suppressors were not let-7 targets, indicating their involvement by other means. Encouraged by this finding, the present inventors tested diverse nucleases for their ability to suppress let-7 and found that xrn-2, the C. elegans orthologue of the yeast 5′-to-3′ exonuclease Rat1p, was a particular potent suppressor of vulva bursting, with >95% (n=3) of animals surviving. By contrast, depletion of the C. elegans homologues of Rex1p through Rex4p 3′-to-5′ exonucleases, regulators of miRNA stability in plants, did not suppress let-7 lethality (Ramachandran 2008).

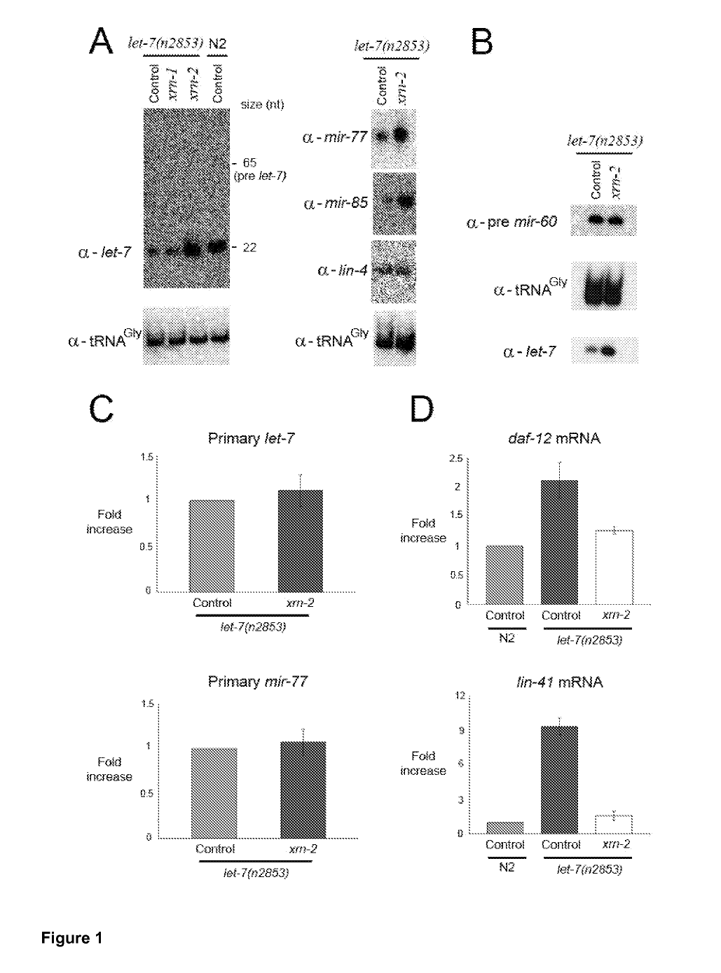

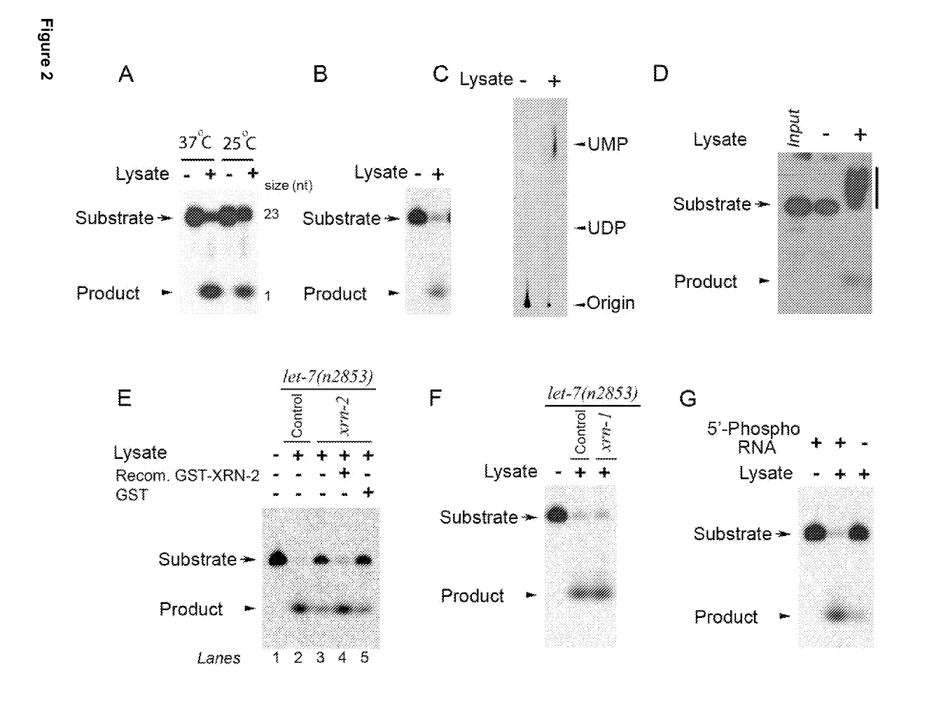

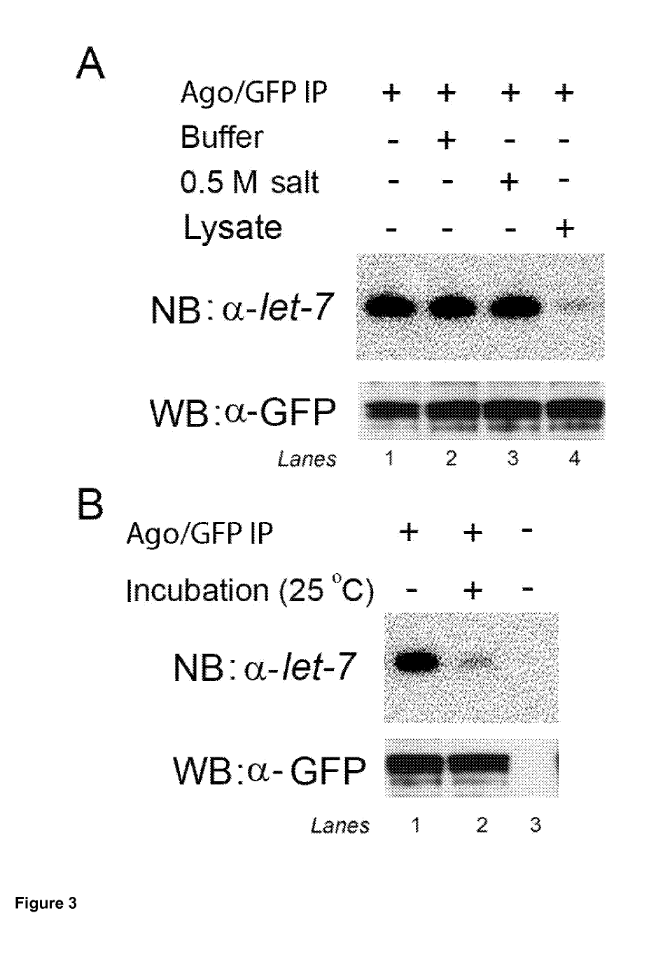

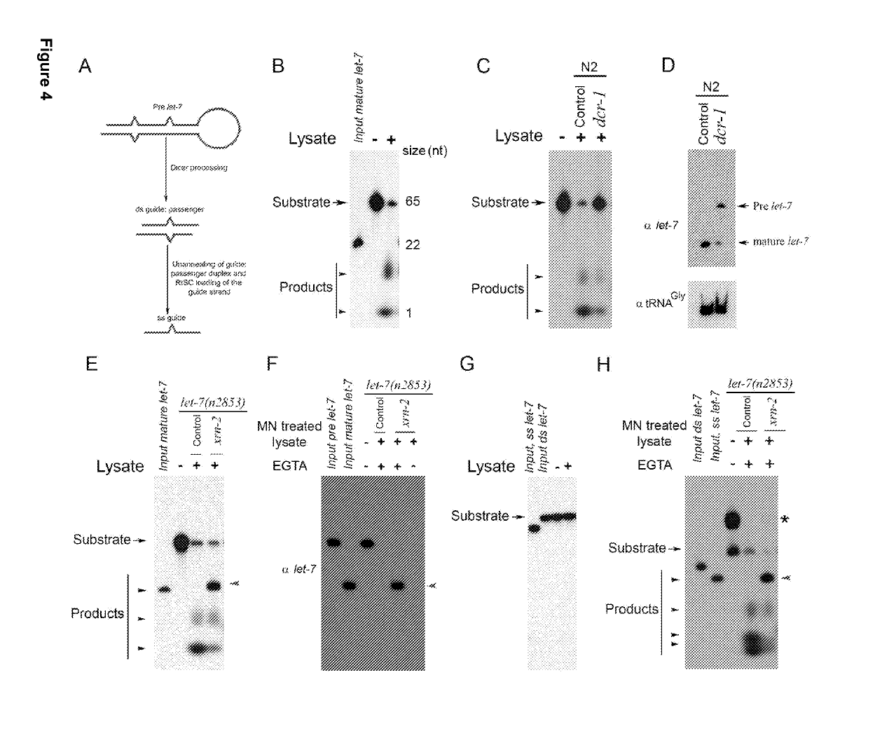

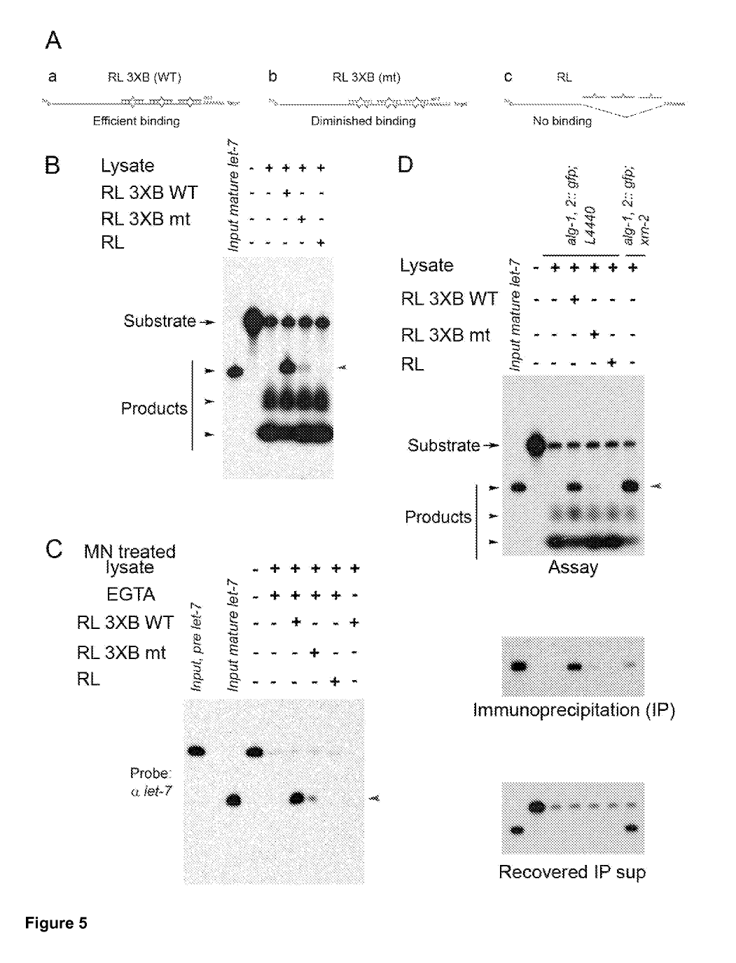

To confirm that depletion of xrn-2 affected let-7 RNA levels, the present inventors examined RNA from late L4 stage let-7(n2853) worms, exposed to either xrn-2(RNAi) or mock RNAi. Consistent with a function in turnover of mature let-7 and/or its precursors, northern blot analysis revealed that mature let-7 levels were increased substantially upon xrn-2 depletion (FIG. 1A).

In addition to its involvement in the processing of rRNA and snoRNA precursors, Xrn2p/Rat1p also functions in quality control of mature tRNA, removing tRNAs that have been incompletely modified. However, the effects of xrn-2 depletion were not restricted to the mutant let-7 miRNA, but were also seen for wild-type let-7, as well as other miRNAs, unrelated in sequence (FIG. 1A and data not shown), demonstrating a general function of XRN-2 in miRNA homeostasis, as opposed to a ‘quality control’ turnover system. Nonetheless, lin-4 was one miRNA that did not appreciably accumulate upon xrn-2 knock down (FIG. 1A, right panel), suggesting the possibility that lin-4 might not be a substrate of xrn-2.

To test whether XRN-2 acts during the miRNA biogenesis pathway to affect pri-miRNA or pre-miRNA levels, the present inventors examined expression of pre-mir-60, which is detectable by northern blotting (Lee & Ambros 2001). Also in this case the present inventors observed that xrn-2(RNAi) did not increase pre-miRNA levels relative to the control situation (FIG. 1B). They also measured the levels of primary miRNAs for pri-let-7 and pri-mir-77 using RT-qPCR, and again found no change in their levels (FIG. 1C) in xrn-2(RNAi) relative to control worms. The present inventors hence concluded that depletion of xrn-2 preferentially, possibly exclusively, affects accumulation of mature miRNAs.

To their knowledge, this was the first time that XRN-2 has been implicated in the turnover of functional, mature RNA species.

The present inventors also noticed that some let-7(n2853); xrn-2(RNAi) animals displayed defects in vulval morphogenesis, i.e., the vulva did not close during the late L4/young adult stage (Fig. S2). Moreover, and as reported previously for xrn-2(RNAi) single mutant animals (Frand 2005), the double mutant animals displayed moulting defects. Vulval formation appeared to be delayed, rather than terminated, as most let-7(n2853); xrn-2(RNAi) adult animals ultimately developed fully closed vulvae and still did not burst. Moreover, xrn-2 depletion not only suppressed the bursting phenotype, but also permitted generation of adult alae, a cuticular structure whose formation depends on let-7 function. Although these observations indicate that suppression of let-7 bursting by xrn-2 depletion is direct and specific, the present inventors sought to confirm that xrn-2(RNAi) enhances let-7 activity by examining the levels of daf-12 and lin-41 mRNAs. These two mRNAs are targets of let-7 (Slack 2000, Grosshans 2005) and loss of let-7 activity increases their transcript levels (Bagga 2005; FIG. 1). Depletion of xrn-2 decreased the levels of these mRNAs comparable to what was seen in the let-7 wild-type situation (FIG. 1D), demonstrating a molecular basis for specific suppression of let-7 by xrn-2(RNAi). These data also confirm that XRN-2 antagonizes functional let-7, rather than acting as a ‘scavenger’ enzyme that clears away inactive miRNA.

XRN-2 is a functional component of the turnover machinery in vitro. To examine miRNA turnover biochemically and in more detail, the present inventors developed an in vitro system using larval lysates and radiolabelled synthetic or in vitro transcribed miRNAs. Initially, pCp labelling was used to block the 3′ terminal hydroxyl group of synthetic let-7, precluding the activity of 3′-to-5′ exonucleases on the substrate. In wild-type worm lysate the substrate was converted to mononucleotides without the production of any visible intermediates both at 25° C., the physiological temperature, and 37° C. (FIG. 2A). As the latter temperature yielded more product, and for technical convenience, the present inventors performed all subsequent in vitro reactions at 37° C. A 5′ labelled synthetic let-7 and an internally labelled let-7 in vitro transcript having free 3′ hydroxyl-groups were similarly degraded, when exposed to the lysate (FIG. 2B). Thin-layer chromatography identified the product as nucleotide-5′-monophosphate, when internally labelled let-7 was used as substrate (FIG. 2C), revealing a hydrolytic mode of degradation as expected of 5′-to-3′ exonucleases (Stevens 1995.). Nuclease activity was largely sequence independent, as other synthetic miRNAs were similarly degraded (data not shown). Nonetheless, the degradative activity was distinct from that seen for other small RNAs such as tRNAs bearing a free 3′-hydroxyl group, for which the present inventors observed an array of bands of sizes greater than the substrate and a final product that was a few nucleotides long (FIG. 2D), reflecting the well-documented degradation by the exosome following adenylation by TRAMP (LaCava 2005).

To examine whether the exonuclease activity depended on xrn-2, the present inventors prepared extract from worms exposed to xrn-2(RNAi). Under these conditions, miRNA degradation was significantly reduced relative to a control RNAi extract (FIG. 2E). By contrast, and mirroring the in vivo situation, depletion of xrn-1 did not affect miRNA turnover (FIG. 2F). The present inventors confirmed that the effect of reducing xrn-2 was specific, as add-back of bacterially expressed, recombinant GST-tagged XRN-2, but not GST alone, partially restored miRNA turnover (FIG. 2E, compare lanes 4 and 5). Consistent also with the known preference of (yeast) Xrn2p/Rat1p for a 5′ phosphate on its substrate (Stevens 1995, 1987), miRNA turnover was decreased when non-phosphorylated substrate was used (FIG. 2G). These results demonstrate that the exonuclease activity working in the lysates on miRNAs is XRN-2.

Pre-miRNA processing and mature miRNA turnover are coupled. The in vivo assays had indicated that XRN-2 specifically affected accumulation of the mature miRNA, but not its precursors. To confirm this in vitro, the present inventors generated radiolabelled pre-let-7 by in vitro transcription, and incubated it with the lysate. This substrate was converted into several products including mononucleotides (FIG. 3B), which is the sole product formed in a mature miRNA turnover assay. To rule out nonspecific degradation of pre-let-7, the present inventors repeated the assay with dcr-1(RNAi) extract, and observed stabilization of the pre-let-7 substrate (FIG. 3C), as they did for the endogenous pre-let-7 in vivo (FIG. 3D). Thus, product formation in the control lysate depended on an upstream processing activity, i.e. the dicing activity of Dicer When pre-let-7 was incubated in xrn-2(RNAi) lysate, the pre-let-7 still disappeared, mirroring the in vivo data and suggesting that xrn-2 does not degrade pre-let-7. Surprisingly, however, the present inventors observed in the xrn-2(RNAi), but not the mock RNAi lysate, accumulation of a band co-migrating with a synthetic mature let-7 (FIG. 3E). The present inventors identified this band as the mature let-7 by performing a scaled-up assay with cold substrate and subjecting the extracted RNA to northern analysis (FIG. 3F). In this experiment, lysates were pre-treated with micrococcal nuclease (MN) to exclude the possibility of detecting endogenous RNAs in our northern analysis. Taken together, these findings show that the assay faithfully recapitulates pre-let-7 cleavage by Dicer and that processing by Dicer is a prerequisite for miRNA degradation by XRN-2. Extraction of RNA under non-denaturing conditions followed by native gel analysis directly demonstrated that the mature let-7 generated by pre-let-7 processing in xrn-2(RNAi) lysates was single stranded (FIG. 3H). The in vitro system described herein thus recapitulates several steps in miRNA biogenesis and turnover, i.e., pre-miRNA processing by Dicer, unannealing of the guide-passenger duplex, and degradation of the single stranded miRNA.

Stabilization of RISC-bound miRNA by target RNA binding. The observation that single-stranded mature miRNA was subject to turnover, whereas a guide:passenger duplex was not, suggested that target binding might modulate miRNA stability. To test this possibility, the present inventors supplemented their lysates with in vitro transcribed let-7 target RNA, i.e., a luciferase coding sequence fused to a 3′ artificial UTR containing three let-7 binding sites or control transcripts with mutated let-7 binding sites or lacking the 3′ UTR entirely (FIG. 4A). Under these conditions, the transcript with the 3′ UTR containing the let-7 binding sites, but not the two control transcripts, efficiently prevented mature let-7 miRNA degradation (FIG. 4B). Importantly, the controls exclude the possibility that excess exogenous RNA simply quenched RNase activity. Northern blot of a scaled-up assay using cold substrate confirmed accumulation of mature let-7 under these conditions (FIG. 4C). Hence, miRNA targets can modulate the extent of mature miRNA degradation in vitro.

To test whether mature miRNA produced by in vitro dicing became associated with Ago, the present inventors prepared lysates from transgenic worms expressing gfp-tagged versions of both of the C. elegans miRNA argonautes, alg-1 and alg-2 (subsequently named GFP/AGO) (Grishok 2001).

Following incubation with radiolabelled pre-let-7, and immunoprecipitation of GFP/AGO, co-immunoprecipitated RNA was extracted, resolved on a gel, and subjected to autoradiography. No radiolabelled RNA, precursor or mature, was detected in GFP/AGO immunoprecipitates from control extracts lacking miRNA target, consistent with the complete degradation of the radiolabelled substrate in the extract. However, addition of the let-7 target RNA permitted not only accumulation of the mature miRNA in the extract (FIG. 4D, top panel), but also its co-immunoprecipitation with GFP/AGO (FIG. 4D, middle panel) demonstrating incorporation of the mature miRNA into miRISC.

let-7 is released from miRISC prior to degradation. Depletion of xrn-2 caused substantial accumulation of mature let-7 generated from pre-let-7 by GFP/AGO larval lysates (FIG. 4D, top panel). However, little mature let-7 co-immunoprecipitated with GFP/AGO (FIG. 4D, middle panel) in the absence of target RNA, whereas abundant let-7 remained in the post-immunoprecipitation (IP) supernatant (FIG. 4D, bottom panel). These data support the notion that miRNAs are dislodged from ALG-1/2 through a mechanism that, in vitro, is modulated by the target RNA binding status of the miRNA, but only partially dependent on XRN-2. Indeed, as both the miRNA 5′ and 3′ ends are thought to be directly bound by Argonaute (Wang 2008), they would be inaccessible to exonucleases while residing in RISC. However, the fact that not only the levels of endogenous miRNAs but also let-7 activity are increased in xrn-2 mutant animals, demonstrates that release and degradation steps are tightly coupled in vivo.

Although these data provide strong support for release of miRNAs from RISC, this result is nevertheless unexpected as the related human siRNA:AGO complexes have been shown to be highly stable (Martinez 2004). To demonstrate such miRNA release from AGO directly, the present inventors investigated the association of GFP/AGO with endogenous miRNA. To this end, they immunoprecipitated GFP/AGO and incubated the protein, while bead bound, with either their assay buffer (AB), AB supplemented with KCl to a final concentration of 0.6 M, or the same amount of lysate from which they had been immunoprecipitated. After recovery, RNA was extracted and probed for presence of endogenous, mature let-7 by northern blot analysis. Addition of neither buffer nor high salt diminished let-7 binding relative to the control, consistent with the reported stability of human AGO-siRNA complexes (Martinez 2004) (FIG. 5A, compare lanes 1, 2, and 3). By contrast, incubation with wild-type larval lysate resulted in a strong loss of let-7 signal, consistent with its removal from the ALG-1/-2 complexes (FIG. 5A lane 4).

Finally, the present inventors immunoprecipitated GFP/AGO from larval lysate either immediately or following 15 minutes of incubation at the worm physiological temperature, 25° C. Consistent with a miRNA release factor acting on RISC, let-7 levels were decreased in immunoprecipitate obtained after the incubation step relative to the pre-incubation immunoprecipitate (FIG. 5B, compare lane1 and 2). As the levels and integrity of GFP/AGO are not altered under these conditions, proteolytic degradation of GFP/AGO does not mediate this release.

### REFERENCES

Filipowicz, W., Bhattacharyya, S, N. & Sonenberg, N. Mechanisms of post-transcriptional regulation by microRNAs: are the answers in sight? Nat. Rev. Genet. 9, 102-114 (2008).
Lim, L. P., Lau, N. C., Weinstein, E. G., Abdelhakim, A., Yekta, S., Rhoades, M. W., Burge, C. B. & Bartel, D. P. The microRNAs of Caenorhabditis elegans. Genes Dev. 17, 991-1008 (2003).
Houbaviy, H. B., Murray, M. F. & Sharp, P. A. Embryonic stem cell-specific MicroRNAs. Dev. Cell 5, 351-358 (2003).
Neilson, J. R., Zheng, G. X., Burge, C. B. & Sharp, P. A. Dynamic regulation of miRNA expression in ordered stages of cellular development. Genes Dev. 21, 578-589 (2007).
Martinez, N. J. et al. Genome scale spatiotemporal analysis of C. elegans microRNA promoter activity. Genome Res. Epub ahead of print (2008).
Thomson, J. M. et al. Extensive post-transcriptional regulation of microRNAs and its implications for cancer. Genes Dev. 20, 2202-7 (2006).
Obernosterer, G., Leuschner, P. J., Alenius, M. & Martinez, J. Post-transcriptional regulation of microRNA expression. RNA 12, 1161-7 (2006).
Chang, T. C. & Mendell, J. T. microRNAs in vertebrate physiology and human disease. Annu. Rev. Genomics Hum. Genet. 8, 215-39 (2007).
Esquela-Kerscher, A. & Slack, F. J. Oncomirs—microRNAs with a role in cancer. Nat. Rev. Cancer 6, 259-69 (2006).
Büssing, I., Slack, F. J. & Grosshans, H. let-7 microRNAs in development, stem cells and cancer. Trends Mol. Med. 14, 400-9 (2008).
Reinhart, B. J. et al. The 21-nucleotide let-7 RNA regulates developmental timing in Caenorhabditis elegans. Nature 403, 901-6 (2000).
Vella, M. C., Choi, E. Y., Lin, S. Y., Reinert, K. & Slack, F. J. The C. elegans microRNA let-7 binds to imperfect let-7 complementary sites from the lin-41 3′UTR. Genes Dev. 18, 132-137 (2004).
Bagga, S. et al. Regulation by let-7 and lin-4 miRNAs results in target mRNA degradation. Cell 122, 553-563 (2005).
Abbott, A. L., et al. The let-7 MicroRNA family members mir-48, mir-84, and mir-241 function together to regulate developmental timing in Caenorhabditis elegans. Dev. Cell 9, 403-14 (2005).
Ding, X. C., Slack, F. J. & Grosshans H. The let-7 microRNA interfaces extensively with the translation machinery to regulate cell differentiation. Cell Cycle 7, 3083-90 (2008).
Ramachandran, V. & Chen, X. Degradation of microRNAs by a family of exoribonucleases in Arabidopsis. Science 321, 1490-92 (2008).
Petfalski, E., Dandekar, T., Henry, Y. & Tollervey D. Processing of the precursors to small nucleolar RNAs and rRNAs required common components. Mol. Cell. Biol. 18, 1181-89 (1998).
Johnson, A. W. Rat1p and Xrn1p are functionally interchangeable exoribonucleases that are restricted to and required in the nucleus and cytoplasm, respectively. Mol. Cell. Biol. 17, 6122-30 (1997).
Grishok, A. et al. Genes and mechanisms related to RNA interference regulate expression of the small temporal RNAs that control C. elegans developmental timing. Cell 106, 23-34 (2001).
Ketting, R. F. et al. Dicer functions in RNA interference and in synthesis of small RNA involved in developmental timing in C. elegans. Genes Dev. 15, 2654-59 (2001).
Lee, R. C. & Ambros V. An extensive class of small RNAs in Caenorhabditis elegans. Science 294, 862-4 (2001).
Frand, A. R., Russel, S. & Ruvkun, G. Functional genomic analysis of C. elegans molting. PLoS Biol. 3, e312 (2005).
Slack, F. J. et al. The lin-41 RBCC gene acts in the C. elegans heterochronic pathway between the let-7 regulatory RNA and the LIN-29 transcription factor. Mol. Cell. 5, 659-69 (2000).
Grosshans, H., Johnson, T., Reinert, K. L., Gerstein, M. & Slack, F. J. The temporal patterning microRNA let-7 regulates several transcription factors at the larval to adult transition in C. elegans. Dev. Cell 8, 321-30 (2005).
Stevens, A. & Poole, T. P. 5′-exoribonuclease-2 of Saccharomyces cerevisiae. Purification and features of ribonuclease activity with comparison to 5′-exonuclease-1. J. Biol. Chem. 270, 16063-69 (1995).
LaCava, J. et al. RNA degradation by the exosome is promoted by a nuclear polyadenylation complex. Cell 121, 713-24 (2005).
Stevens, A. & Maupin, M. K. A 5′-3′ exoribonuclease of human placental nuclei: purification and substrate specificity. Nucleic Acids Res. 15, 695-708 (1987).
Wang, Y., Sheng, G., Juranek, S., Tuschl, T. & Patel, D. J. Structure of the guide-strand-containing argonaute silencing complex. Nature 456, 209-13 (2008).
Martinez, J. & Tuschl, T. RISC is a 5′ phosphomonoester-producing RNA endonuclease. Genes Dev 18, 975-80 (2004).
Bhattacharyya, S, N., Habermacher, R., Martine, U., Closs, E. I. & Filipowicz W. Relief of microRNA-mediated translational repression in human cells subjected to stress. Cell 125, 1111-24 (2006).
Kedde, M. & Agami, R. Interplay between microRNAs and RNA-binding proteins determines developmental processes. Cell Cycle 7, 899-903 (2008).
Reinhart, B. J. et al. The 21-nucleotide let-7 RNA regulates developmental timing in Caenorhabditis elegans. Nature 403, 901-6 (2000).
Hutvágner, G., Simard, M. J., Mello, C. C. & Zamore, P. D. Sequence-specific inhibition of small RNA function. PLoS Biol. 2, E98 (2004).
Grosshans, H., Johnson, T., Reinert, K. L., Gerstein, M. & Slack, F. J. The temporal patterning microRNA let-7 regulates several transcription factors at the larval to adult transition in C. elegans. Dev. Cell 8, 321-30 (2005).
Ding, X. C., Slack, F. J. & Grosshans H. The let-7 microRNA interfaces extensively with the translation machinery to regulate cell differentiation. Cell Cycle 7, 3083-90 (2008).
Lee, R. C. & Ambros V. An extensive class of small RNAs in Caenorhabditis elegans. Science 294, 862-4 (2001).
Pall, G. S. & Hamilton, A. J. Improved northern blot method for enhanced detection of small RNA. Nat. Protoc. 3, 1077-84 (2008).
Hager, D. A. & Burgess R. R. Elution of proteins from sodium dodecyl sulfate-polyacrylamide gels, removal of sodium dodecyl sulfate, and renaturation of enzymatic activity: results with sigma subunit of Escherichia coli RNA polymerase, wheat germ DNA topoisomerase, and other enzymes. Anal Biochem. 109, 76-86 (1980).
Chatterjee, S. et al. An RNA-binding respiratory component mediates import of type II tRNAs into Leishmania mitochondria. J. Biol. Chem. 281, 25270-7 (2006).
Kolb, F. C. et al. Human dicer: purification, properties, and interaction with PAZ PIWI domain proteins. Methods Enzymol. 392, 316-36 (2005).
Pillai, R. S. et al. Inhibition of translational initiation by let-7 MicroRNA in human cells. Science 309, 1573-6.
Matranga, C., Tomari, Y., Shin, C., Bartel, D. P. & Zamore P. D. Passenger-strand cleavage facilitates assembly of siRNA into Ago2-containing RNAi enzyme complexes. Cell 123, 607-20 (2005).
Dziembowski, A., Lorentzen, E., Conti, E. & Séraphin, B. A single subunit, Dis3, is essentially responsible for yeast exosome core activity. Nat. Struct. Mol. Biol. 14, 15-22 (2007).
Lee, M. H. & Schedl, T. Identification of in vivo mRNA targets of GLD-1, a maxi-KH motif containing protein required for C. elegans germ cell development. Genes Dev. 15, 2408-20 (2001).

1. A method for modulating miRNA, said method comprising modulating XRN2.

2. The method of claim 1 wherein the method is performed in a subject and wherein an effective amount of a modulator of XRN2 is administered to said subject.

3. The method of claim 2 wherein the method is performed to treat a disease and wherein a therapeutically effective amount of said modulator of XRN2 is administered to said subject.

4. The method of claim 3, wherein the disease is a cancer, a metabolic disease, a developmental disorder, a cardiac disease or a viral infection.

5. The method of claim 2, wherein the modulator of XRN2 is a small molecule.

6. The method of claim 2, wherein the modulator of XRN2 is an antibody.

7. The method of claim 2, wherein the modulator of XRN2 is an agonist.

8. The method of claim 2 wherein the modulator of XRN2 is an inhibitor.

9. The method of claim 8 wherein said inhibitor of XRN2 decreases or silences the expression of XRN2.

10. The method of claim 9 wherein the inhibitor is a siRNA.

11. The method of any of claim 2 wherein the subject is a mammal.

12. (canceled)

13. (canceled)

14. A method for the identification of a substance that modulates the expression of XRN2 and/or its biological activity, which method comprises the steps of:
(i) contacting a XRN2 polypeptide or a fragment thereof having the biological activity of XRN2, a polynucleotide encoding such a polypeptide or polypeptide fragment, an expression vector comprising such a polynucleotide or a cell comprising such an expression vector, and a test substance under conditions that in the absence of the test substance would permit XRN2 expression and/or biological activity; and
(ii) determining the amount of expression and/or biological activity of XRN2, to determine whether the test substance modulates biological activity and/or expression of XRN2,
wherein a test substance which modulates biological activity and/or expression of the XRN2 is a potential therapeutical agent to treat cancer.

15. The method of claim 14, wherein the biological activity of XRN2 is the degradation of mature miRNA.

16. The method of claim 5 wherein the small molecule is a RNase inhibitor.

17. The method of claim 11 wherein the mammal is a human.


SEQUENCE LISTING (28 sequences)

SEQ ID NO: 1 - DNA, 22 bp, Caenorhabditis elegans
aactatacaacctactacctca

SEQ ID NO: 2 - DNA, 22 bp, Caenorhabditis elegans
aactatacaacctactatctca

SEQ ID NO: 3 - DNA, 22 bp, Caenorhabditis elegans
tggacagctatggcctgatgaa

SEQ ID NO: 4 - DNA, 24 bp, Caenorhabditis elegans
gcacgacttttcaaatactttgta

SEQ ID NO: 5 - DNA, 21 bp, Caenorhabditis elegans
tcacacttgaggtctcaggga

SEQ ID NO: 6 - DNA, 42 bp, Caenorhabditis elegans
cttgaactagaaaatgtgcataatatcacgtactttgtcatg

SEQ ID NO: 7 - DNA, 40 bp, Caenorhabditis elegans
caaccctgaaccagacgtaccaactggaggcccagttggt

SEQ ID NO: 8 - DNA, 27 bp, Caenorhabditis elegans
gcttggaaggcatccatgctgaccatt

SEQ ID NO: 9 - DNA, 23 bp, Caenorhabditis elegans
tcctagaacacatctccctttga

SEQ ID NO: 10 - DNA, 20 bp, Caenorhabditis elegans
cgcagcttcgaagagttctg

SEQ ID NO: 11 - DNA, 20 bp, Caenorhabditis elegans
cattgttcgtttcgctttca

SEQ ID NO: 12 - DNA, 24 bp, Caenorhabditis elegans
ccaataactgattcaacattccaa

SEQ ID NO: 13 - DNA, 20 bp, Caenorhabditis elegans
gatcctccgatgaacgaaaa

SEQ ID NO: 14 - DNA, 20 bp, Caenorhabditis elegans
ctcttcggcttcaccagaac

SEQ ID NO: 15 - DNA, 19 bp, Caenorhabditis elegans
ggattgttcgacaccaacg

SEQ ID NO: 16 - DNA, 22 bp, Caenorhabditis elegans
accatgatgtcaaactgctgtc

SEQ ID NO: 17 - DNA, 20 bp, Caenorhabditis elegans
gatcccgagtacccacaaga

SEQ ID NO: 18 - DNA, 20 bp, Caenorhabditis elegans
ccaccaccacctctcacata

SEQ ID NO: 19 - DNA, 33 bp, Caenorhabditis elegans
gaaagaattcatgggagttcccgcattcttcag

SEQ ID NO: 20 - DNA, 38 bp, Caenorhabditis elegans
gaaagcggccgcgattatctccatgatgaatttccgtg

SEQ ID NO: 21 - DNA, 87 bp, Caenorhabditis elegans
gtaatacgactcactatagggagactactacctcactgatgagtccgtgaggacgaaacggtacccggtaccgtctgaggtagtagg

SEQ ID NO: 22 - DNA, 34 bp, Caenorhabditis elegans
aactatacaacctactacctcagacggtaccggg

SEQ ID NO: 23 - DNA, 87 bp, Caenorhabditis elegans; identical to SEQ ID NO: 21

SEQ ID NO: 24 - DNA, 77 bp, Caenorhabditis elegans
ggtaaggtagaaaattgcatagttcaccggtggtaatattccaaactatacaacctactacctcagacggtaccggg

SEQ ID NO: 25 - DNA, 25 bp, Caenorhabditis elegans
gaattctaatacgactcactatagg

SEQ ID NO: 26 - DNA, 22 bp, Caenorhabditis elegans
aactatacaacctactacctca

SEQ ID NO: 27 - DNA, 25 bp, Caenorhabditis elegans
gaattctaatacgactcactatagg

SEQ ID NO: 28 - DNA, 22 bp, Caenorhabditis elegans
ggtaaggtagaaaattgcatag